SEQUENCE LISTING (19 sequences)

SEQ ID NO: 1 - DNA, 20 bp, Artificial Sequence
  Synthetic sequence
cacctggccttcatacacct

SEQ ID NO: 2 - DNA, 20 bp, Artificial Sequence
  Synthetic Sequence
agaaaggaagggccaagaaa

SEQ ID NO: 3 - DNA, 20 bp, Artificial Sequence
  Synthetic sequence
gaccgtgtccttaccgtgac

SEQ ID NO: 4 - DNA, 20 bp, Artificial Sequence
  Synthetic sequence
cctgtgtcgcctttaccact

SEQ ID NO: 5 - DNA, 18 bp, Artificial Sequence
  Synthetic sequence
gattctgtgtaccgccgg

SEQ ID NO: 6 - DNA, 18 bp, Artificial Sequence
  Synthetic sequence
tccaccgacgtgccaggc

SEQ ID NO: 7 - DNA, 19 bp, Artificial Sequence
  Synthetic sequence
tgattctgtgtaccgccgg

SEQ ID NO: 8 - DNA, 20 bp, Artificial Sequence
  Synthetic sequence
tgattctgtgtaccgccgga

SEQ ID NO: 9 - DNA, 19 bp, Artificial Sequence
  Synthetic sequence
gtccaccgacgtgccaggc

SEQ ID NO: 10 - DNA, 20 bp, Artificial Sequence
  Synthetic sequence
gtccaccgacgtgccaggcc

SEQ ID NO: 11 - DNA, 14110 bp, Artificial Sequence
  Synthetic sequence
gcggccgccatggacgagcaggcgggtcccggcgtcttcttcagcaacaaccacccgggcgccggcggtgccaaggggctcgggcctctggcggaggctgccgcggccggcgacggggcggctgcggcgggggcggcccgagcccagtacagtctcccggggatcctgcacttcctgcagcacgagtgggcccgcttcgaggtggagagagcccagtgggaggtggagcgggcggagctgcaggcccagattgccttcctgcagggagaaaggaagggccaagaaaatttgaagaaggatcttgtgaggaggatcaaaatgttggagtatgctcttaaacaggaaagagccaaataccacaagttgaaatacgggacagaattgaatcagggagatatgaagcctccaagctatgattctgatgaaggtaatgaaacagaagtgcagccacaacaaaacagccagttaatgtggaaacaaggtcgacaactactcagacagtatctacaggaggtgggttatacagatactattctagatgtgaaatctaaacgagtgcgagctttgttgggcttttcaagtgatgtcacggacagggaagatgacaaaaatcaggactcagttgtaaatggcacagaggctgaagttaaagagacagcaatgattgcaaaatctgagttaacagattctgcctccgtgctggataatttcaaattccttgaaagtgcagctgcagatttcagtgatgaagatgaagatgatgatgttgatggaagagagaaaagcgtcattgatacttcaacaattgttaggaaaaaagcattgcctgacagcggtgaagatcgagatacaaaagaagctctaaaggagtttgacttcttggttacatcagaggaaggagacaatgaatctagaagtgcaggcgatggaacagactgggaaaaggaagaccagtgtctcatgcctgaagcctggaatgtggaccagggagtaattaccaaactcaaggaacaatacaaaaaggagagaaaggggaaaaagggggtgaagaggcccaataggtcaaaactacaagatatgcttgctaatttgagagatgttgatgaacttccttcattgcagccatctgtgggttcaccttccagacccagcagctccaggcttcctgaacatgaaattaatagggcagatgaagtggaagcattgacatttcctccttcttctggaaagtcattcatcatgggagcagatgaagcccttgaaagtgaactgggacttggagaactagcaggccttacggtggccaatgaagcagactcactaacttatgatatagcaaacaataaagatgcattgaggaagacatggaaccctaagtttacattgagaagtcactttgatggcatccgagcccttgctttccatcccattgagcctgttttgataacagcatcagaggatcacacattaaaaatgtggaatttacagaaaacagccccagccaaaaagagcacttctcttgatgtagaacctatctatacattcagagcccataaaggtccagtgctttgtgtggtaatgagcagcaatggtgagcagtgttacagtggtggtactgatggactgatccagggctggaataccactaatcccaacatcgacccctatgattcttatgatccttctgttttacgaggccctctgctaggccacacggatgcagtctggggtttggcttatagtgcagcacatcagcgtttgttgtcctgttcagcagatggcactctgcgtttatggaatacaactgaggttgctccagcactaagtgtatttaatgatactaaagaactgggaatccctgcctctgtggatctagtgagcagtgacccgagccatatggtagcatcattcagcaagggatatacaagcatttttaacatggaaacacaacaacgcattctcactttagaatccaatgtagatacaacagccaactcttcctgccaaataaatagagtcatcagtcatcctactcttccgatcagcatcactgctcatgaagacaggcacatcaaattctatgataacaatacaggcaaactgatccactcgatggtagcccacctagaagctgttacaagtttagcagttgatcccaatggcctttacttgatgtctggcagtcatgactgttcaatacgtttatggaatctagaaagtaagacgtgtatccaagaattcacagctcatcgaaaaaagtttgaagaatcgattcatgatgtagctttccacccatccaaatgctatatagccagtgctggagctgacgcactggctaaagtctttgtatgacgcaatgcatcatcttcaccttctagctgtttataagtaatcaactgcacacaagagatacagaagacgagggcaagaatcatctcgtcctgcccttttgttctgctgaaggagcacagagaacatttgttgaagtatagttttgcaattcatatactgttttctaaaactaaggtttgttcaggttgctgcaagctcagctgaatctgtgagcctgaggtctgtttcaaatttctccccaataggcgcctttatttctgaggtggttctaattcgctaggcaggcctgagcgaatacaagtttagcttgtccctgttgagtaagtagggcatgctacaatggataatttaaaagcttgatagctgggactgaatagaagaaaacgggaaacttagaccaagttctcccctgagaaatcttgttaaaacacattaagtagtcaaaatagtaatctttatcatatctgccatattaacccctttgtgtataatgaccaggcagtgttaaactgagtttttaatttgactctcctttcagctgttcacataaagcacctggcaaagcattttacctgttagggggagaaaaatgcaataatatgttaaatatattattattcagaaatgctagacaaatagtttgcaagcagatttctatattgtattacagttttgaaaacagatttggtatcattctaaagtttagctatcaagcactcaccattgtgaagaatagttgatcgactctgcttttactaacagatttaacttattcaggtttgaaattctgtttatttttaagggtaaacagggcatatctctttaataatttctattttaaaattcactttattcatgtgacatttatagtaccagagtgattatttcataccaatgaaatcttacttttcagtgactgaaaagatagaatttatttgctaataatcatttatcaacacccacctaatcatgcttcagatgatctgtgctctttcttctttgtcacgttcaagttatctgacatctaccaagaacagttcataattattaatagcccaaagagtatggttaacttcacgcagctgcttttgcagaagcactaataaaaacaaatattttgtaccctagaagtcctctattaatccctgtaagaatctcctggaaacctggatgaaagagtttaatgccaaatattggctaaaaagttctgtctttctctctccctattcctcccccattgtttaattgagatatacatgttttaatgggcagtggatcagtattgtttctggggaaatccacaccttaaaaggccaggcttccccagcccaacttacctgtgacccacctacatgtgttcaccttttgggattctgaagaccctgctatctagacctagtctcctgtcataaagacattatttctatagtggtttatgtctctcttactctttgtcccaaatgacagagaccaatctagaaattggaaataaattattaacgtccaactttccaccaaaaaacatataaataggctcttagattttaaaaatattaaatctatagatattttaaccaattaaaacaatttctacatattccattccccacattttccatcgtcataacaatgatgcttctcatgatacgtacatctactccttgaaaaagtattctggtggtggtgtggctaatgattgacaaatatttctgtaaaatttctgcctgtttgcctctagaattctgcattatacaggcaaattttctttaaagctagagacttgtgctaaatttgcctgattttcatacagatatgctgtagtaatacagctaccttttcatttcatctttgtgctttggttaacaagcaggctcaagagactgctttgtgtaattaaatgaattgacctaaacatgagctgtcaggtgaatacttaatatttccatgcagatacagaattccatattgtgttcagtaaatttcttttggttcttcacagtgaagaaagaatgtttctaacctaacatttgtagaaggatactggaaaattccttccaggaaggaaggaaggaagatgccattacttggtgtatttatgatggatgaccacaaagctattgttctaccagttattaattaccttctaagatcttcagttactgctttattaatgtcactgaaattctaaaaaccaggacattcttcccccgttcattttatgcttcatggttgaaatttttctgtcagtgagtggaaaataaccatgagatacatagactgtagcgcaaatggaactgttgaaagttccttagaaagccagtgctgtggcattggagactacggagagagtcttctctaccagcactgcatgatctcagcctagaaatgagccaaagctgtcttccaaccagagtagcatatacatgtgtctgacttttcccctagcctgttgaacattcaatgaaaattcacagaatgaacaaattcaatgtttaaacagccattgaaatgctttcatagtgcttcatttgtaaatattttgtttgaaatgtattggaatagtatgaaaattagcgggaggtttggatccctgccttattttcatatgctgtggatacatctctgggagcttgcagcacccttctcaaaactcaactgtcatatggagctcatttgctactcaaaccatagacagattatttaacaaaagtgatataatgaccttcagtatttttctattgaacaagtttaacaacttttgccattaaacaaatacttagttatgcaaaatatttcgcttttataatcattttagttatgactaaaacgggggaaattgagctgcatcactgatgatgaactttgtgagaaattttttttttaatctgactttcattccttcagtattcttgctataaatttcttttggagataatgatgaagatttatttgaaaatgtctaaaatgtatgtatattttataaatatatattatatatattgtaagaatatatataatatataaactatatatatatatatatatatatatatatatatatatatataaaccataggtgcattttactgttttgtattttcatttttggaggtagtatacacagcagataatgatgagtatgatgagtgttgtgctgtttgcttttgcagtataatatatagttctaagtgtttaaattactgtatatacaatattcattataggctagcatgcttgttttaaagactgtcattctagtgtattaaaatctgaatgtaagaaaacctggctattcaaatgtgaattgaaaaatattttattctcagtactgaactatttccattgaacattcagactttttaacaaaaacaaatatcatcaagaaggcaatagctagaaaatgagaggtgcctttaaaaaaaaaaatgcagccttacagtgaggatgaagattgagtttggggagggggtgggagggggggcacagagagagagagagagttagtgtgtgtgtgtgtgtgtgtgtgtgtgtgtgtgtgtgcatgcatgcgtgtgtgtgtgtgtgtgtgtgtttaccaatctgtgaaggtcctgatttgtgaccatcaatgtctttttaatgtatctgctcaacttctaggacttcaggggacatttcatgcgctttgtgctcttctgagcaccatatatactttccaagaattttgaatgtgaattcctagaaatttcagtagagaaaacaatagctattctttaattatagtaaagttcaggtagaaggagaaattatttgttcaccaaaattatgggatctcattaatgttggctaagtaaatgtaattttttaaaggctcactttttaatacttcatatatttccctcttacaaaacagcatgtcatgaagttcttatgtggtaaagattcaactagtaaatatttttaggttaaatacctgaactagttaaaattccacctaagaatagtcttaaaatatttaaaaagcatctctcatatctacctctttgtcattgctgccattttaactgaagcaaatctttatgacatctgaaatgatggttggatgcaattaaaaaaaaatctatgttggtgcttttctcaggctttgtcatttaagttatattgtattaagatgtcacaaatcagactgaaaggcttagaattaattttgtttttctttgaaagaattgcttaaaattaaccatcaatgtttttagcgagtggagataatgaatctttggttggtgtctgtctgtgtgatgttttcaaccaatagccaaataattattgaacttacatgaaaaagagtgctaagtataattttatttagattactttattctttgaaagtgactacaaggctattaagtacataatcattcattttatttactcagaagttgcctctccattactgttaacttgtttagtaatttatataattgcagtacttttaagtgttggacactgggattcaggttgtatacaaagtagtagctgacgaaaaatatgtatatttttgtgaaatgtatcaagccctaaagttcattttagagaaaggcccagaaaaacgtatctgtgtttaaaaacaaaggacaggtcattcgtcaatttgaatcatttttacttttgccactacaaactttttaattggcaatatccatcatccaactggatcttcatgtcagtgtcagggatgccaggttgacaaagtatttaacagatcctttcagtttttgttttgtttttttaattctaaaaaaaacaaatgttaggccaaacagatccctagatcccactcattgattctggcggtattcctaaagtggtgcttagggggtcagaattttctggatctttgctaatccaagctttagatttaatttaaccaggaccacatgcttgtcatctctctgatgcaaattttcaaaatcattttaatttagattctaatgtctgcctgggtttttaacaggctgtgaaccagtgagtgccttgttaatgtagaatgatttttcccccctgggtgggtgttagttagctcctctctgaaaatgtctcagagaatggtattttaattgacttgtagagagctgctaaatttgttattatcagcaatcagaaagtctttggtctaccacacatctccctccctactatctcttcattgtttggctgaagtttgcctcttgtgagaaaacaatacaactccccttctccacacactttttttccagcccagattaataggtatcccctaccctaatcccaactctatgatctaataccttttttaggaggaaccacttctttttaatattgaaatctgctggttactttatgttttctcaagttcagttgactagagctgaccacagggtgtcatcaattcagattcctaaataaccctttctctccacacccagtcctccagctggtgagccctgggccagattgtttgatcttcaggtattttaaattttagttacatcaattaatgtaaattaatccaaatgctaatttttgtggtgcaagaagtttcttatgtaaattcagggtatggataagctaattaagatatccatctttggtggctctaagtgtattatttgtttttaaataaagtgtacaaatatagataacatgctataggtgtctcatgaattggaatatttgtcagatttgggatctagctgtggctttacagtaatcagttaaattaacatagtctgatttggggcttttgggggaggagttgtagaggggtgcagagaaataaaaggatattagacaaagtatacatgagtccaaaattaatgaaccagtcttaattactgattgtgaggaaggagaagtttatagagaatttgggcctctgaaaagccaaattaaaatagcttctaggattgggtcagtggtactttcagagacacatatgtttaatgaataggttttatgtttttcaaatgttcagtttaaatggcttttaaaatttatacacatatataaatacaatgtttttattttttgtttcgctacttaacagacatacacaagccttccacactccctgccccttcaaatcaacattatttttcaaggcctttggcctgcccagaactcacagtctttttgttccaatcaggatccacaatacaatatgaaatttaaaaggcatctcttctgatatccttgaaactatccaccattgtttggtgttctgtatgctggtatactaatgagtattatatcctttggtatgtacacacattttcagtagtgttggaaacacctcttttatctttttaatgagaactgagacatgattagcttgagtaattttggagatctatctcaggaggtcattaaaactccaaaaattattcacggattgatgaacctcctggggtcagcctaagattgccaggtagtattctgacctcctctgggtttcccaagatcactgatgttttgcagggccccagcattgtcctggcacataaaatattcccagaacaccttgggagtcaagcataccttttaacctaaacattataatcatggagcatccctccgcctgttacttctcaggatgaccagaacactccagttctctaaggaagcactgacatacggctctgaaaattggaatccgttttctgaaacacaaattttaactcagtaagttttgtatatcaaatgattcaaaacccaccaaacataaaatgactcgcttctaagtatcactcaaaccctacaaaacctaaccctttccttattcctaattgatatttcactaaaggtaatggtagtttcagttagattggaagtcattatttgggtgagcaaagtctgcaataacctactgataggaaggacctatttcatagaaaagtatcttgtccttggtgatttagccattcaccttacgtgaagagtggtccagatgccagctccacattgactttttctgtgtccctctttaggtaattaacccagtgatgtaaaggagagcccacagtaaaacatctgttaaacagcacttggagtactatagtgttattaatttatgtgctataatttcttatgccatcataaaatctagtgtatgtaccaggttcccaattcattgcctcaaacaaaagatttttaaaaattctaatagcatcaaattaagggtaatggtagggaaacccatcatccacaattaaatatgcccaaactgaaaactttaagaaaaaaaagcacatggaaagcaacgttttacattaatcgtgagttgtagaaataaacagcttgtggtttgtatatgtgccgaaatgttaaagtatgctggcatccaactacttccctttggaaatgggaccaagacaataatttttcatgcataaacaaaattaacttagagcaaaagtaattttatcagaaacagtttattttgggggctggataacttgggtgagagtgtggggaaagaagtaccttaccaaaaaggagaagcaactgaccctgtggcctgagccacattgtcttccatttcaacttcaatgggctggcagtataccacttctgacctcaaagaatgaatggttccaattctggcttgtcattggtccttgttatctaaattaaatatttttaggaaatatatcaaaagtatcctagagccccatggcaaagtgtcagaggaaatagttttcattatattttaggaagctgtaaaaatataagcccaagtattttgtgtcatctgcatatgtcaggatgaagaccaggcatgtaagaaatatcctaaagtagccaagtgataatctcatgaaaaaatatgagaatcgtttttacagagtgagttctcttttgaatggttttgactatgcttttaaaaacatttttaaaatgtacttacatctttttcgatagcccacgtatttcagaatatcctcttgatagaataatatcactcagtgtgatttttagaaaaagaaaaactcggtggtctcatatcttttgacagttgtttgtgaataataccctccccaacaaccttcccagtactcaactgctatgtaagaatgctttcttatgtggtaaatgtctcagtattttgctgcctggtatttgttcagtttccttgtatatctcagggtcagaaggaatcaggctttctcccaactctgaaacattcagacttactttctttttggtcagccttttaacaagcaagacaataaactccttttgtcagaatcgatttgattaaaaaaaaaaaaaaagaatgtgctccttatttctcattgctgtagaatacgtaggaatacacatctactaatcacaattaaaaaataagagaatctttagaaacatcattgctttctggaggtagaaattttgaacttgaaggttgtagttccaggcaattttgtgcccttgaggtgtccaattgatagtatagcttgcctctgctaataaatacacaaaagttttctttcctgtgattttcagaaattatttttggaatacaaagattaataaggaatatgtgatgtttatctcctgcatttcaaaatcttctggattttttttgaggcatgaggagcaggtaatgagattttttttctgaatatcttgattctctcccttaacccagattttgcatattatgcattgctcatttgtcacctacaaaaataaaggttaatgattatttgtttgcttttgtctatataagctccctaaggcacagaccatgttcgtttttcttatgttcctcttagcactgtgcctggtgccttgccatataaaaggtaccctataaatatttgttggctgagtaatcagagagcccatttcaaaatctaaattatacaaatcatcaaagaccaaaaaaaaaaaaaaaaaatctcttgacagttgatgctcaaatgcatttggttctgtggagcttatcctgagattgaggtctgcaccttgactggagaggaatttggactttacctcacaggtagcaacaaaatgcctgagtagcaacgtctttatactgcacagttccagtcctgcctgcgtgaaaactgagcagagcatggtggggacaggaaggtgtccctaaaccctgttgatgacctcccatctatccccggacccccatatctcctctgctcatcccctgttccccactggcctctgatcagttgaaagggagcttttctagcctggaacctgagcctgccttgattatcctgggctcagaacaggtccacttccacactctttccttgcaacctacagatggccctactgactgctgttgtccaggagaaagacgaacctcccagtggaatcttgtcaggaaaggcatccctctggggagctttctttggagaggaaaattctgggacaaagttcctcaggatgctctcaggctggtttttgtcttgaagattttatagtaattgtaccaatgtttattaactctcctaggaacatctctgtctttggttttctgacagcactccagcagggcccagggctttagctgaacagtgcaattgttttaggaataaaacagtcaacaagttagagaccaactatttatatttaatattcaaatgactttaaatcattaaaagaatcagcaaattacatatgaaatattcaacgaataatctcaacattaaacatataccatgtaagaactgtcattaaaattactaaaaatatgattcctttaagatcagttcaacacagaagaaagcaggaatttggcctggacaataggacatttattgtctgttagattttagacaagtcaactcttctaaattggcttctcatctatgaaatggggataatagatctgcctaattaggttgatttgagataacggatgtaaaacacctaataaaataagcatccagcaaatgacttgttcactctctgaatacagacttctggtcatgtagttcggtcacactcctgatagcttctagaacatcagggttttaggagaaataggttaaaatgaacatcaagagtagcagaagactactttccttaatatttgagtgccagctatatgccacacagttctagttgctggcagtcagcagtaaacaaaaacttcacctgatgcagcacattccaggtgggggaacaagtgaaaatatagggaatattagtgataagtgcagagggaaaaaaaatagagcagggaagggggatgatttgatgtttttcaggttaagaaagtgacagaaatgaatttaaatgcaaaaaacaatgcataaagattcataatggcaaaacgaagcatgtcccatcataccccttcattcctacctccctgatatgtatctttctaattattcatacaaatgtgtatacccagttattacatatggggttttattctacatattatgctgcacttgctttttctaatataatgtgtatcatgagcaccttacaaatttttaaaggtctctcaaatttatcagtaaacatatacagtctaacgttaatgagaagatattttggtgggcagtttgctttaaataaatgggatcatgtacgttctctgcatctcacatttctcatttaacaatgtaggatgagaatttctctaagtctatcatagctcttatttcctttttattattacaataaacattcctgtgtatatgtttgtcaccatgatggttttacgtctgcggaatagattccctggagtaggattgctgcattgaggagtatgtatatttaaatttttaaataaatattgacagattgtttcccaaagtggccggaacagtttttatttccatcagtaaatgtataaaaacacttttcccttaatactcatggcaataactattgtaacattaaaaaatacgtagaaaaaagaaaaaaatcctggtggatgtagtgatttttcagtgttattttaatttccattttcctgactactataaatgtagacatcttttcaaatgttttggtcatctggattgcactcagaactgaattatctattcaaagttctgtaggttagaactccagggaaactcaactgggctgtctgttcaaggcccaacaaggctgaaatgaaagtgtcttccagggtgggtactcaactaaagctctgaggaggaattcgcttccagcctcattaaagtcagaggatggaggtccctgtctcctagctgactgtcaaatctgatcctttagaggttgctccgtggtccttgcacgtggtcccttccaccttcaaagcttgccagggtgcatcaaattatttttatactttgactctacttctaccagccagagaaaactgcttttaaagggctcttgtgactagtttaagcacacccagattgttaaggtcaactgattagtagctttaattacatctgccaagtccctctgccctgcaatgtaacatcaaccatggaagtaacatgaggggcagagttcatgggggccatcttagaattctgcctaccacaaatattcgtagttatacttttatatacatttttaaatttttcttatttttggtccatatgtttgtaccgtttgtctttttagttaactagtttgtgaaagcattttgtatgttgtacaatatatgttcagattttcttatcttggatgcaaacatttttttccaactcttgtaagttgtctgttgaccacattcaaaagtactttttccaaataaaactttgccttttgaatttca

SEQ ID NO: 12 - DNA, 6267 bp, Artificial Sequence
  Synthetic sequence
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggatgaatacttgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 13 - DNA, 4310 bp, Artificial Sequence
  Synthetic sequence
gtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccagtctcccgagcggcgcccgcctcccgccggtgcccgcgccgggccgtggggggcagcatgcccgcgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgaggggtccctagcagcccaccctgctgctggtgcacagccactccccggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacgaactttccgaaaaataaagacacctggttgctaacctggaaaaaaaaaaaaaaaaa

SEQ ID NO: 14 - DNA, 2847 bp, Artificial Sequence
  Synthetic sequence
gcgtttgaaactccggcgcgccggcggccatcaagggctagaagcgcgacggcggtagcagctaggcttggcccccggcgtggagcagacgcggacccctccttcctggcggcggcggcgcgggctcagagcccggcaacgggcgggcgggcagaatgagtctgcaggtcttaaacgacaaaaatgtcagcaatgaaaaaaatacagaaaattgcgacttcctgttttcgccaccagaagttaccggaagatcgtctgttcttcgtgtgtcacagaaagaaaatgtgccacccaagaacctggccaaagctatgaaggtgacttttcagacacctctgcgggatccacagacgcacaggattctaagtcctagcatggccagcaaacttgaggctcctttcactcaggatgacacccttggactggaaaactcacacccggtctggacacagaaagagaaccaacagctcatcaaggaagtggatgccaaaactactcatggaattctacagaaaccagtggaggctgacaccgacctcctgggggatgcaagcccagcctttgggagtggcagctccagcgagtctggcccaggtgccctggctgacctggactgctcaagctcttcccagagcccaggaagttctgagaaccaaatggtgtctccaggaaaagtgtctggcagccctgagcaagccgtggaggaaaaccttagttcctattccttagacagaagagtgacacccgcctctgagaccctagaagacccttgcaggacagagtcccagcacaaagcggagactccgcacggagccgaggaagaatgcaaagcggagactccgcacggagccgaggaggaatgccggcacggtggggtctgtgctcccgcagcagtggccacttcgcctcctggtgcaatccctaaggaagcctgcggaggagcacccctgcagggtctgcctggcgaagccctgggctgccctgcgggtgtgggcacccccgtgccagcagatggcactcagacccttacctgtgcacacacctctgctcctgagagcacagccccaaccaaccacctggtggctggcagggccatgaccctgagtcctcaggaagaagtggctgcaggccaaatggccagctcctcgaggagcggacctgtaaaactagaatttgatgtatctgatggcgccaccagcaaaagggcacccccaccaaggagactgggagagaggtccggcctcaagcctcccttgaggaaagcagcagtgaggcagcaaaaggccccgcaggaggtggaggaggacgacggtaggagcggagcaggagaggacccccccatgccagcttctcggggctcttaccacctcgactgggacaaaatggatgacccaaacttcatcccgttcggaggtgacaccaagtctggttgcagtgaggcccagcccccagaaagccctgagaccaggctgggccagccagcggctgaacagttgcatgctgggcctgccacggaggagccaggtccctgtctgagccagcagctgcattcagcctcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctgacctccacggagccgctgtccccgcccccctgctcccgtctgtctgtcctgtctgattctcttaggtgtcatgttcttttttctgtcttgtcttcaacttttttaaaaactagattgctttgaaaacatgactcaataaaagtttcctttcaatttaaacactgaaaaaaaaaaaa

SEQ ID NO: 15 - DNA, 64 bp, Artificial Sequence
  Synthetic sequence
agatcggaagagcacacgtctgaactccagtcacnnnnnnatctcgtatgccgtcttctgcttg

SEQ ID NO: 16 - DNA, 64 bp, Artificial Sequence
  Synthetic sequence
caagcagaagacggcatacgagatnnnnnngtgactggagttcagacgtgtgctcttccgatct

SEQ ID NO: 17 - DNA, 20 bp, Artificial Sequence
  Synthetic sequence
cacacctgggaaaggaccta

SEQ ID NO: 18 - DNA, 18 bp, Artificial Sequence
  Synthetic sequence
cattggaggcataagctg

SEQ ID NO: 19 - DNA, 20 bp, Artificial Sequence
  Synthetic Sequence
agcacggtaacgtagggtgt

(19) United States
(12) Patent Application Publication
(10) Pub. No.: US 2016/0186269 A1
(43) Pub. Date: Jun. 30, 2016

(54) NOVEL TRANSLOCATIONS IN LUNG CANCER

(71) Applicants: STICHTING HET NEDERLANDS KANKER INSTITUUT-ANTONI VAN LEEUWENHOEK ZIEKENHUIS, Amsterdam (NL); AGENDIA N.V., Amsterdam (NL)

(72) Inventors: Rene Bernards, Amsterdam (NL); Ian Jordan Majewski, Amsterdam (NL); Lorenza Mittempergher, Amsterdam (NL)

(21) Appl. No.: 14/893,991

(22) PCT Filed: May 27, 2014
(86) PCT No.: PCT/NL2014/050338

§ 371 Date: Nov. 25, 2015

(30) Foreign Application Priority Data
May 27, 2013 (NL) PCT/NL2013/050384

(51) Int. Cl. C12Q 1/68

(52) U.S. Cl. CPC C12Q 1/6886; C12Q 2600/156

(57) ABSTRACT

The invention relates to methods for determining the presence or absence of striatin-anaplastic lymphoma kinase (STRN-ALK) gene fusion and/or a Fibroblast Growth Factor Receptor 3—transforming acidic coiled-coil containing protein 3 (FGFR3-TACC3) gene fusion in an individual, especially an individual suffering from lung cancer. The invention further relates to a method for diagnosing an individual as having adenocarcinoma, and to a method for treating said individual. The invention additionally relates to a method for diagnosing an individual as having squamous cell carcinoma, and to a method for treating said individual.

### FIELD

The invention relates to the field of cancer. In particular, the invention relates to the diagnosis and prognosis of patients having adenocarcinoma, especially adenocarcinoma of the lung.

Recurrent translocations have been studied in leukemia for over half a century (Nowell and Hungerford, 1960. J Natl Cancer Inst 25: 85), but in the past decade it has become clear that structural rearrangements and fusion genes also contribute to the development of solid tumours. Sometimes these rearrangements are very common, consider fusions involving ETS-family members in prostate cancer (Tomlins et al., 2005. Science 310: 644), but many seem to occur at a low frequency, and often involve multiple fusion partners, which represents a significant challenge for discovery and for subsequent diagnostic screening.

Concerted and systematic efforts have been applied to define the key genetic alterations that drive lung cancer (Ding et al., 2008. Nature 455: 1069; The TCGA research network, 2012, Nature 489: 519; Imielinski et al., 2012. Cell 150: 1107; Peifer et al., 2012. Nature Genetics 44: 1104; Rudin et al., 2012. Nature Genetics 44: 1111; Seo et al., 2012. Genome Res 22: 2109). Numerous technologies have been employed, including exome sequencing, whole genome sequencing and transcriptome sequencing. These studies have shown that the genomic landscape of lung cancer is highly complex, due to high rates of somatic mutations, copy number alterations and genetic rearrangements. Although this work has highlighted many new driver genes, our knowledge of the genetic rearrangements and fusion genes that occur in lung cancer remains limited, because only a small handful of genome sequences have been completed.

The best-characterized fusion gene in lung cancer is EML4-ALK, which was discovered using a cell-based transformation assay (Soda et al., 2007. Nature 448: 561). ALK has since been found to be involved in a variety of fusions, all of which preserve its kinase domain. Recent clinical trials have demonstrated that tumours that carry ALK fusions respond to the small molecule ALK inhibitor crizotinib (Shaw et al., 2011. Lancet Oncol 12: 1004). It took a little over five years between the identification of ALK as a therapeutic target in lung cancer and its validation in a genotype-driven clinical trial. The results obtained with ALK, and other fusion kinases such as BCR-ABL, serve as an important reminder that cancer cells become addicted to signaling through oncogenic kinases and that there is tremendous value in identifying these events and developing therapeutic strategies to target them.

While candidate based approaches have been applied successfully to define new fusion kinases in lung cancer, such as the identification of RET and ROS1 fusions in adenocarcinoma (Bergethon et al., 2012. J Clin Oncol 30: 863; Takeuchi et al., 2012. Nat Med 18: 378), a global method for detection is needed to fully understand the diversity of kinase alterations driving the disease. We developed a high-throughput platform for systematically profiling kinase fusions that relies upon specific enrichment of kinase transcripts. Using this approach we screened a panel of non-small cell lung cancer (NSCLC) samples and identified a number of activating mutations, amplifications and novel fusion transcripts. The novel fusion transcripts will provide much needed insight into the oncogenic pathways operating in lung cancer and make a strong case for applying specific inhibitors for treatment of lung cancers comprising these fusion transcripts.

The invention therefore provides a method for determining the presence or absence of striatin-anaplastic lymphoma kinase (STRN-ALK) gene fusion and/or a Fibroblast Growth Factor Receptor 3—transforming acidic coiled-coil containing protein 3 (FGFR3-TACC3) gene fusion in an individual, said method comprising a) evaluating a relevant nucleic acid sample of said individual to determine whether a portion of STRN nucleic acid is adjacent to a portion of ALK nucleic acid on a single polynucleotide, and/or whether a portion of FGFR3 nucleic acid is adjacent to a portion of TACC3 nucleic acid on a single polynucleotide; and b) identifying said individual as having a STRN-ALK gene fusion when a portion of the STRN nucleic acid is adjacent to a portion of the ALK nucleic acid on a single polynucleotide, and/or as having a FGFR3-TACC3 gene fusion when a portion of the FGFR3 nucleic acid is adjacent to a portion of the TACC3 nucleic acid on a single polynucleotide.

The term “individual”, as is used herein, refers to a human. An individual can be a patient, especially a patient that is suffering from cancer, including adenocarcinoma and especially lung cancer, more specifically non-small cell lung cancer. Lung cancer accounts for about 15% of all diagnosed cancers in human and causes the most cancer-related deaths in both men and women (source: Cancer facts and FIGS. 2007, American Cancer Society). The three main types of primary lung cancers are mesothelioma, small cell lung cancer, and non-small cell lung cancer. Mesothelioma is a rare type of cancer which affects the covering of the lung (the pleura). It is often caused by exposure to asbestos. Small cell lung cancer (SCLC), also called oat cell lung cancer, is characterized by the presence of small cells that are almost entirely composed of a nucleus. SCLC frequently occurs in (ex)smokers and is quite rare for people that never smoked. SCLC tends to spread early in development of the tumor and is often treated with chemotherapy rather than surgery. Non-small cell lung cancer (NSCLC) is the most common form of lung cancer and is diagnosed in about 85% of all lung cancer patients. NSCLC represents a diverse group of cancers with the main groups being squamous cell carcinoma, adenocarcinoma, and large cell carcinoma. Other, minor groups comprise pleomorphic carcinoma, carcinoid tumor, salivary gland carcinoma, and unclassified carcinoma. Adenocarcinoma is the most common subtype of NSCLC, accounting for 50% to 60% of NSCLC.

The term “nucleic acid” or “polynucleotide” refers to single stranded or double stranded deoxyribonucleic acid (DNA), ribonucleic acid (RNA), and copy DNA (cDNA) that is reverse transcribed from RNA, preferably from messenger RNA (mRNA). Said DNA preferably comprises or is chromosomal (genomic) DNA, which includes, for example, coding regions, introns, 5′ and 3′ untranslated regions, promoter/enhancer regions, and intergenic DNA.

The term “a portion of a nucleic acid of gene A” refers to a nucleic acid of which at least about 20 nucleotides, at least about 25 nucleotides, at least about 30 nucleotides, at least about 40 nucleotides, at least about 50 nucleotides, at least about 100 nucleotides, at least about 250 nucleotides, at least about 500 nucleotides, at least about 1,000 nucleotides, at least about 2,000 nucleotides, at least about 5,000 nucleotides, are of a gene A. The term “are of gene A” indicates that the nucleic acid sequence of said portion of a nucleic acid is homologous or identical to a nucleic acid sequence of gene A. Said homologous or identical sequence may encompass the coding region, one or more introns, 5′ and/or 3′ untranslated regions, promoter/enhancer regions and intergenic DNA. The term “homologous”, as used herein, indicates that the nucleotide sequence is at least 90% identical to a nucleotide sequence of gene A.

For example, the term “a portion of STRN nucleic acid” refers to a nucleic acid of which at least about 20 nucleotides, at least about 25 nucleotides, at least about 30 nucleotides, at least about 40 nucleotides, at least about 50 nucleotides, at least about 100 nucleotides, at least about 250 nucleotides, at least about 500 nucleotides, at least about 1,000 nucleotides, at least about 2,000 nucleotides, at least about 5,000 nucleotides, are homologous or identical to a nucleotide sequence selected from the coding region, one or more introns, 5′ and/or 3′ untranslated regions, promoter/enhancer regions and intergenic DNA of STRN.

The term “adjacent to ” in the context of a STRN-ALK gene fusion indicates that a portion of STRN nucleic acid is directly joined (fused) to a portion of ALK nucleic acid on a single polynucleotide, or that said portions of STRN and ALK nucleic acids are separated from each other on a single polynucleotide by less than 100, 90, 80, 70, 60, 50, 40, 30, 25, 20, 15, 10, 9, 8, 7, 6, 5, 4, 3, or 2 nucleotides. Nucleotides that separate portions of STRN nucleic acid and ALK nucleic acid may be non-homologous to chromosome 2.

The term “adjacent to ” in the context of a FGFR3-TACC3 gene fusion indicates that a portion of FGFR3 nucleic acid is directly joined to a portion of TACC3 nucleic acid on a single polynucleotide, or that said portions of FGFR3 and TACC3 nucleic acids are separated from each other on a single polynucleotide by less than 100, 90, 80, 70, 60, 50, 40, 30, 25, 20, 15, 10, 9, 8, 7, 6, 5, 4, 3, or 2 nucleotides. Nucleotides that separate portions of FGFR3 nucleic acid and TACC3 nucleic acid may be non-homologous to chromosome 4.

The term “STRN” refers to Striatin, Calmodulin Binding Protein, also termed SG2NA. STRN encodes a protein of 780 amino acid residues which has C-terminal WD repeats. A putative caveolin-binding domain-encoding region is located between nucleotides 172 and 198 of NM_003162.3, a putative coiled coil domain-encoding region is located between nucleotides 217 and 357 of NM_003162.3, and a putative calmodulin binding domain-encoding region is located between nucleotides 455 and 357 of NM_003162.3 (Castets et al., 2000. J Biol Chem 275: 19970). STRN is located on cytogenetic location 2p22.2. The mRNA of STRN is provided by Reference Sequence (RefSeq) NM_003162.3, which is depicted in FIG. 4A.

The term “ALK” refers to Anaplastic Lymphoma Kinase. ALK encodes a protein of 1620 amino acid residues which has a tyrosine kinase domain in the C-terminal half from nucleotides 4298-5101 of NM_004304.4. ALK is located on cytogenetic location 2p23. The mRNA of ALK is provided by Reference Sequence (RefSeq) NM_004304.4, which is depicted in FIG. 4B. A

The term “FGFR3” refers to Fibroblast Growth Factor Receptor 3. FGFR3 encodes a protein of 806 amino acid residues which has a tyrosine kinase domain in the C-terminal half of the protein from amino acids 472-748. FGFR3 is located on cytogenetic location 4p16.3. The mRNA of FGFR3 is provided by Reference Sequence (RefSeq) NM_001163213.1, which is depicted in FIG. 4C. A kinase domain is located between nucleotides 1670-2499 of NM_001163213.1.

The term “TACC3” refers to Transforming, Acidic, Coiled-Coil-containing protein 3. TACC3 encodes a protein of 838 amino acid residues which has coiled coil structures close to the C-terminus at amino acids 641-725 and 754-838. TACC3 is located on cytogenetic location 4p16.3. The mRNA of TACC3 is provided by Reference Sequence (RefSeq) NM_006342.2, which is depicted in FIG. 4D.

Said portion of the STRN gene that is included in a STRN-ALK fusion gene preferably comprises exons 1-3, corresponding to nucleotides 1-421 of NM_003162.3, or a relevant part thereof. This region includes the caveolin binding domain-encoding region and the coiled coil encoding region, but excludes the Ca2+/calmodulin-binding domain-encoding region and the C-terminal WD-domains-encoding region.

Said portion of the FGFR3 gene that is included in a FGFR3-TACC3 fusion gene preferably comprises exons 1-18, and comprises a kinase encoding domain. Said portion of the FGFR3 gene preferably comprises nucleotides 1-2536 of NM_001163213.1, or a relevant part thereof.

Said portion of ALK nucleic acid that is included in a STRN-ALK fusion gene preferably comprises exons 21-29 of ALK, and preferably includes a major part or all of exon 20. Said portion of ALK nucleic acid preferably comprises a kinase-encoding region. Said portion preferably is from nucleotide 4126 to end of NM_004304.4, or a relevant part thereof.

Said portion of TACC3 that is included in a FGFR3-TACC3 fusion gene preferably comprises nucleic acid comprises a coiled coil domain-encoding region, preferably both coiled coil domain-encoding regions. Said portion of TACC3 preferably comprises exon 10-16 of TACC3, more preferably the C-terminus-encoding part from nucleotide 1992 to end of NM_006342.2, or a relevant part thereof.

The term “a relevant nucleic acid sample” refers to a nucleic acid sample that comprises nucleic acid from cancer cells, or that is suspected of comprising nucleic acid from cancer cells. Said relevant nucleic acid sample is preferably derived from a bodily fluid, for example blood, pleural fluid or sputum, more preferably from a part of a cancerous growth or from a growth that is suspected to become cancerous. Said cancerous growth is preferably removed by surgical treatment prior to obtaining said nucleic acid sample. The act of removing the cancerous growth is not part of the present invention. Said cancerous growth may have been frozen directly after isolation and stored at a temperature below 0° C. As an alternative, said cancerous growth was fixed, for example by formalin, and stored. Methods for isolating nucleic acid from cells, including cancer cells, are known in the art. It is preferred that at least 10% of the cells from which the relevant nucleic acid sample is derived are cancer cells or suspected to be cancer cells, more preferred at least 20%, and most preferred at least 30%. Said percentage of cancer cells can be determined by analysis of a stained section, for example hematoxylin and eosin-stained section, from the cancerous growth. Said analysis can be performed or confirmed by a pathologist.

The bodily fluid and/or a cancerous growth is preferably directly used in a method of the invention, or stored under protective conditions that preserve the quality of the nucleic acid. Examples of such preservative conditions are fixation using e.g. formaline, the use of RNase inhibitors such as RNAsin™ (Pharmingen) or RNAsecure™ (Ambion), and the use of preservative solutions such as RNAlater™ (Ambion) and RNARetain™ (Assuragen). It is further preferred that said preservative condition allows storage and transport of said tissue sample at room temperature. A preferred preservative condition is the use of RNARetain™ (Assuragen).

The nucleic acid sample that is evaluated in a method of the invention preferably comprises genomic DNA or mRNA. Extracted mRNA is preferably converted into complementary DNA (cDNA) using a reverse-transcriptase enzyme and nucleotides, as is known to a skilled person. Methods for isolating genomic DNA or mRNA from a bodily fluid and./or a cancerous growth are known in the art and include, for example, commercial kits such as, but not limited to, QIAamp™ mini blood kit, Agencourt Genfind™, Roche Cobas® Roche MagNA Pure® or phenol : chloroform extraction using Eppendorf Phase Lock Gels®, and the NucliSens extraction kit (Biomerieux, Marcy l'Etoile, France). In other methods, mRNA may be extracted using MagNA Pure LC mRNA HS kit and Mag NA Pure LC Instrument (Roche Diagnostics Corporation, Roche Applied Science, Indianapolis, Ind.). Other published protocols and commercial kits are available including, for example, Qiagen products such as the QiaAmp DNA Blood MiniKit (Qiagen, Valencia, Calif.), the QiaAmp RNA Blood MiniKit (Qiagen, Valencia, Calif.); Promega products such as the Wizard Genomic DNA Kit (Promega Corp. Madison, Wis.), Wizard SV Genomic DNA Kit (Promega Corp. Madison, Wis.), the SV Total RNA Kit (Promega Corp. Madison, Wis.), PoIyA Tract System (Promega Corp. Madison, Wis.), or the PurYield RNA System (Promega Corp. Madison, Wis.).

A preferred method according to the invention comprises amplification of at least part of the extracted nucleic acid. Known amplification methods include, but are not limited to, nucleic acid sequence based amplification (NASBA), strand-displacement amplification, loop-mediated isothermal amplification and polymerase chain reaction such as multiplex PCR and multiplex ligation-dependent probe amplification. Said amplification preferably is by PCR.

Said amplification preferably amplifies a relevant part of nucleic acid of STRN and ALK, and/or of FGFR3 and TACC3. Said amplification preferably employs a primer that is specific for a relevant part of STRN and ALK, and/or of FGFR3 and TACC3. A primer is specific when it comprises a continuous stretch of nucleotides or nucleotide analogues that are complementary to a nucleotide sequence of a nucleic acid of said gene, or a cDNA product thereof. A primer is additionally specific when it comprises a continuous stretch of nucleotides or nucleotide analogues that are partially complementary to a nucleotide sequence of a nucleic acid of said gene, or a cDNA product thereof. Partially means that a maximum of 2 nucleotides in a continuous stretch of at least 20 nucleotides differ from the corresponding nucleotide sequence of a nucleic acid or cDNA of said gene, more preferred a maximum of 1 nucleotide in a continuous stretch of at least 15 nucleotides differs from the corresponding nucleotide sequence of a nucleic acid or cDNA of said gene. The term complementary is known in the art and refers to a sequence that is related by base-pairing rules to the sequence that is to be detected. It is preferred that the sequence of the primer is carefully designed to minimize nonspecific hybridization to said primer. It is preferred that the primer is or mimics a single stranded nucleic acid molecule. The length of said complementary continuous stretch of nucleotides can vary between 15 nucleotides and 100 nucleotides, and is preferably between 16 nucleotides and 30 nucleotides, more preferred between 18 and 25 nucleotides, and most preferred about 20 nucleotides.

A primer for amplification of a relevant part of nucleic acid of STRN preferably is or mimicks a nucleotide sequence that is located in exons 1-3 of STRN, preferably a nucleotide sequence selected from nucleotides 1-421 of NM_003162.3. Said primer is directed towards the 3′ end of said relevant part of nucleic acid of STRN.

A primer for amplification of a relevant part of nucleic acid of ALK is complementary to a nucleotide sequence that is located in exons 20-29 of ALK, and preferably complementary to a nucleotide sequence selected from nucleotide 4126 to end of NM_004304.4. Said primer is directed towards the 5′ end of said relevant part of nucleic acid of ALK..

A primer for amplification of a relevant part of nucleic acid of FGFR3 preferably is or mimicks a nucleotide sequence that is located in exons 1-18 of FGFR3, preferably a nucleotide sequence selected from nucleotides 1-2536 of NM_001163213.1. Said primer is directed towards the 3′ end of said relevant part of nucleic acid of FGFR3.

A primer for amplification of a relevant part of nucleic acid of TACC3 is complementary to a nucleotide sequence that is located in exons 10-16 of TACC3, and preferably complementary to a nucleotide sequence selected from nucleotide 1992 to end of NM_006342.2. Said primer is directed towards the 5′ end of said relevant part of nucleic acid of TACC3.

Primers can be designed using publicly available software such as, for example, Primer-BLAST, ePrime, and Beacon Designer. Criteria for primer design include, without limitation, length, GC content, and Tm (melting temperature). A primer preferably specifically hybridizes to a target sequence on a relevant part of STRN, ALK, FGFR3 or TACC3.

The term “specific hybridization” indicates that two nucleic acid sequences share a high degree of complementarity. Specific hybridization complexes form under permissive annealing conditions and remain hybridized after any subsequent washing steps Permissive conditions for annealing of nucleic acid sequences are routinely determinable by one of ordinary skill in the art and may occur, for example, at 65° C. in the presence of about 2×SSC. The stringency of hybridization may be expressed, in part, with reference to the temperature under which the wash steps are earned out Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. The term “specific hybridization” does not include hybridization of two nucleic acids which differ over a stretch of 20 contiguous nucleotides by two or more bases, more preferred hybridization of two nucleic acids which differ over a stretch of 15 contiguous nucleotides by one or more bases.

A pair of primers is preferably used for amplification across the fusion break points of STRN-ALK and/or FGFR3-TACC3. Pairs of primers preferably have similar melting temperatures since annealing in an amplification reaction such as PCR occurs for both primers simultaneously. It is further preferred that the length of the fragment that is amplified is between 40 and 2000 nucleotides, preferably between 50 and 1000 nucleotides, preferably between 100 and 800 nucleotides, preferably between 150 and 600 nucleotides.

A preferred primer pair for amplification of the fusion break point of STRN-ALK comprises the nucleotide sequence F: 5′-CACCTGGCCTTCATACACCT (SEQ ID NO: 1) and R: 5′-AGAAAGGAAGGGCCAAGAAA (SEQ ID NO: 2), wherein F denotes a forward primer and R denotes a reversed primer.

A preferred primer pair for amplification of the fusion break point of FGFR3-TACC3 comprises the nucleotide sequence F: 5′-GACCGTGTCCTTACCGTGAC (SEQ ID NO: 3) and R: 5′-CCTGTGTCGCCTTTACCACT (SEQ ID NO: 4).

A further preferred method of the invention comprises detecting a STRN-ALK gene fusion and/or a FGFR3-TACC3 gene fusion by hybridizing a nucleic acid probe encompassing a first portion that is specific for a STRN nucleic acid and a second portion that is specific for an ALK nucleic acid and/or a nucleic acid probe encompassing a first portion that is specific for a FGFR3 nucleic acid and a second portion that is specific for a TACC3 nucleic acid.

A probe comprises a continuous stretch of nucleotides or nucleotide analogues that are complementary to a nucleotide sequence of a nucleic acid of a gene, or a cDNA product thereof. A probe preferably is specific for a relevant part of STRN and ALK, and/or of FGFR3 and TACC3, and preferably encompasses the fusion break point of a STRN-ALK gene fusion and/or FGFR3-TACC3 gene fusion. A probe is specific when it comprises a continuous stretch of nucleotides or nucleotide analogues that are complementary to a nucleotide sequence of a nucleic acid of a gene, or a cDNA product thereof, preferably complementary to the fusion break point of a STRN-ALK gene fusion and/or FGFR3-TACC3 gene fusion. A probe is additionally specific when it comprises a continuous stretch of nucleotides that are partially complementary to a nucleotide sequence of a nucleic acid of a STRN-ALK gene fusion and/or FGFR3-TACC3 gene fusion.

Partially means that a maximum of 1 nucleotide in a continuous stretch of at least 15 nucleotides differs from the corresponding nucleotide sequence of a nucleic acid or cDNA of said gene fusion. The term complementary is known in the art and refers to a sequence that is related by base-pairing rules to the sequence that is to be detected. It is preferred that the sequence of the probe is carefully designed to minimize nonspecific hybridization to said probe. It is preferred that the probe is or mimics a single stranded nucleic acid molecule. The length of said complementary continuous stretch of nucleotides can vary between 20 nucleotides and 10K nucleotides, and is preferably between 50 nucleotides and 2000 nucleotides, more preferred between 100 and 1000 nucleotides, and most preferred about 200 nucleotides.

A preferred probe comprises a nucleotide sequence 5′-GATTCTGTGTACCGCCGG (SEQ ID NO: 5) for detection of a STRN-ALK fusion gene and/or 5′-TCCACCGACGTGCCAGGC (SEQ ID NO: 6) for detection of a FGFR3-TACC3 gene fusion. A further preferred probe comprises a nucleotide sequence 5′-TGATTCTGTGTACCGCCGG (SEQ ID NO: 7) or 5′-TGATTCTGTGTACCGCCGGA (SEQ ID NO: 8) for detection of a STRN-ALK fusion gene and/or 5′-GTCCACCGACGTGCCAGGC (SEQ ID NO: 9) or 5′-GTCCACCGACGTGCCAGGCC (SEQ ID NO: 10) for detection of a FGFR3-TACC3 gene fusion.

A probe is preferably labeled with, for example, an isotope, a fluorescent moiety, a colored substance, allowing detection of the probe by suitable means including spectroscopy, biochemically, immunochemically, or chemical means, such as fluorescence, chemifluoresence, or chemiluminescence, or any other appropriate means. A preferred probe is either directly labeled with a fluorescent probe, for example Rhodamine, Texas Red, Cy2, Cy3, Cy5 or AMCA, or labeled with a reporter molecule, for example biotin, digoxigenin or dinitrophenol for indirect detection methods such as immunohistochemistry. A probe, preferably a labeled probe, is preferably used in Fluorescent In Situ Hybridization (FISH) studies.

A preferred method of the invention comprises determining the presence or absence of said gene fusion by determining the nucleotide sequence of a nucleic acid, preferably the amplified nucleic acid, comprising a fusion break point of a STRN-ALK gene fusion and/or FGFR3-TACC3 gene fusion. The nucleotide sequence of said nucleic acid is preferably determined by dideoxy sequencing, matrix-assisted laser desorption/ionization time-of-flight mass spectrometry, or sequencing by hybridization, including hybridization with sequence-specific oligonucleotides and hybridization to oligonucleotide arrays, as is known to the skilled person.

A preferred method of the invention comprises determining the presence or absence of said gene fusion by determining the size of an amplified nucleic acid comprising a fusion break point of a STRN-ALK gene fusion and/or FGFR3-TACC3 gene fusion. Said size is preferably determined by HPLC, capillary electrophoresis, size exclusion chromatography, and/or agarose gel electrophoresis, as is known to a skilled person.

The invention further provides a method for diagnosing an individual as having adenocarcinoma or squamous cell carcinoma, said method comprising determining the presence or absence of STRN-ALK gene fusion and/or of FGFR3-TACC3 gene fusion according to a method of the invention; and diagnosing said individual as having adenocarcinoma when said STRN-ALK gene fusion is present and/or diagnosing said individual as having squamous cell carcinoma when said FGFR3-TACC3 gene fusion is present. Said individual can be a patient, especially a patient that is suffering from cancer, especially lung cancer , more specifically non-small cell lung cancer. It is preferred that a sample from which a relevant nucleic acid sample is evaluated is removed from the individual prior to obtaining said nucleic acid sample. The act of removing the sample from the individual is not part of the present invention.

The term adenocarcinoma, as is known to the skilled person, refers to a cancer of epithelial tissue that has glandular origin and/or glandular characteristics.

Adenocarcinoma's frequently occur in the lung, prostate, breast, stomach and throat.

The invention also provides a probe according to the invention, for use in a method for diagnosing an individual as having adenocarcinoma or squamous cell carcinoma, especially adenocarcinoma of the lung or squamous cell carcinoma of the lung.

The invention further provides a method for treating an individual suffering from lung cancer, especially adenocarcinoma, said method comprising diagnosing an individual as having adenocarcinoma according to the method of claim 13; and treating said individual with an selective inhibitor of ALK. Known ALK inhibitors include 3-[(1R)-1-(2,6-dichloro-3-fluorophenyl)ethoxy]-5-(1-piperidin-4-ylpyrazol-4-yl)pyridin-2-amine (Crizotinib; Pfizer), AP26113 (2,4-Pyrimidinediamine, 5-chloro-N2-[4-[4-(dimethylamino)-1-piperidinyl]-2-methoxyphenyl]-N4-[2-(dimethylphosphinyl)phenyl]; ARIAD Pharmaceuticals, Inc); LDK378 (C23H28BrN7O3; Novartis); ASP3026 (N2-[2-Methoxy-4-[4-(4-methyl-1-piperazinyl)-1-piperidinyl]phenyl]-N4-[2-[(1-methylethyl)sulfonyl]phenyl]-1,3,5-triazine-2,4-diamine; Astellas Pharma Inc.), CH5424802 (9-Ethyl-6,11-dihydro-6,6-dimethyl-8-[4-(4-morpholinyl)-1-piperidinyl]-11-oxo-5H-benzo[b]carbazole-3-carbonitrile;Hoffmann-La Roche), GSK1838705A (2-(2-(1-(2-(dimethylamino)acetyl)-5-methoxyindolin-6-ylamino)-7H-pyrrolo[2,3-d]pyrimidin-4-ylamino)-6-fluoro-N-methylbenzamide; GSK) and NVP-TAE684 (TAE684; 5-chloro-N4-(2-(isopropylsulfonyl)phenyl)-N2-(2-methoxy-4-(4-(4-methylpiperazin-l-yl)piperidin-l-yl)phenyl)pyrimidine-2,4-diamine; Novartis).

The invention further provides a method for treating an individual suffering from lung cancer, especially SCC, said method comprising diagnosing an individual as having SCC according to the method of claim 13; and treating said individual with an inhibitor of FGFR3. Known FGFR3 inhibitors include NF449 (4,4′,4″,4′″-[Carbonylbis(imino-5,1,3-be-nzenetriyl-bis(carbonylimino))]tetrakis-1,3-benzen-edisulfonic acid, octasodium salt; PKC412 ((9S,10R,11R,13R)-2,3,10,11,12,13-Hexahydro-10-methoxy-9-methyl-11-(methylamino)-9,13-epoxy-1H,9H-diindolo[1,2,3-gh:3′,2′,1′-lm]pyrrolo[3,4-j][1,7]benzodiamzonine-1-one), SU5402 (2-[(1,2-Dihydro-2-oxo-3H-indol-3-yl-idene)methyl]-4-methyl-1H-pyrrole-3-propanoic acid or (Z)-3-(4-methyl-2-((2-oxoindolin-3-ylidene)methyl)-1H-pyrrol-3-yl)propanoic acid), and PD173074 (1-tert-butyl-3-(2-(4-(diethylamino)butylamino)-6-(3,5-dimethoxyphenyl)pyrido[2,3-d]pyrimidin-7-yl)urea), BGJ398 (NVP-BGJ398; 3-(2,6-Dichloro-3,5-dimethoxy-phenyl)-1-{6-[4-(4-ethyl-piperazin-1-yl)-phenylamino]-pyrimidin-4-yl}-1-methyl-urea) and AZD4547 (N-(5-(3,5-dimethoxyphenethyl)-1H-pyrazol-3-yl)-4-((3S,5R)-3,5-dimethylpiperazin-1-yl)benzamide).

The present invention further provides a kit which contains, in an amount sufficient for at least one assay, any of the amplification primers and/or probes, for detecting the presence or absence of a STRN-ALK gene fusion and/or a FGFR3-TACC3 gene fusion in a relevant nucleic acid sample of an individual, especially an individual suffering from long cancer, especially NSCLC. Typically, said kit also includes instructions recorded in a tangible form (e.g., contained on paper or an electronic medium) for using the packaged primers and/or probes in a detection assay for determining the presence of a STRN-ALK gene fusion and/or a FGFR3-TACC3 gene fusion in a test sample.

### FIGURE LEGENDS

FIG. 1. STRN is a novel fusion partner for ALK.

A. ALK fusion genes detected in the NSCLC samples. B. RT-PCR and capillary sequencing confirming the fusion between exon 3 of STRN and exon 20 of ALK (M, marker, W, water, FF, frozen tissue, P, paraffin embedded, N, negative sample). C. FISH was performed with split probes that flank the ALK locus, enlarged sections of the image are show at right. D. Tissue sections were stained with an ALK specific antibody. The STRN-ALK sample is shown (at right), together with a negative sample (at left).

FIG. 2. FGFR3 is activated by multiple mechanisms in SCC.

A. A schematic representation of the FGFR3-TACC3 fusion (Ig-like domains, 2^{nd}, 4^{th} and 5^{th} block; kinase domain, 8^{th} block, TACC domain, 10^{th} block). B. RT-PCR and capillary sequencing was used to demonstrate the fusion between exon 18 of FGFR3 and exon 10 of TACC3. C. Tissue sections were stained with an FGFR3 specific antibody. The sample that carries FGFR3-TACC3 shows high expression of FGFR3 (at right). A sample that was negative for FGFR3 is included as a control (at left). D. FGFR3 S249C was detected through kinome-centred sequencing; a coverage histogram is shown at top for one sample. The mutation was validated with capillary sequencing.

FIG. 3. FGFR3 is highly expressed in a subset of SCCs.

A. Three tissue microarrays were assembled to assess FGFR3 expression across a panel of NSCLCs. Representative samples are shown to demonstrate negative (at left) or positive (at right) staining. B. Of SCC samples, 10/136 were positive, whereas none of the 144 adenocarcinomas were positive. Two additional FGFR3-TACC3 fusion events were detected in samples that were positive by immunohistochemistry.

FIG. 4. RefSeq sequences.

A. RefSeq NM_003162.3. B. RefSeq NM_004304.4. C. RefSeq NM_001163213.1. D. RefSeq NM_006342.2.

### EXAMPLES

### Example 1

Patient Material and Sequencing

The cohort included 95 patients, 80 of which were previously described in a study that developed a prognostic classifier for early stage lung cancer (Roepman et al., 2009. Clin Cancer Res 15: 284). Patient material was available from frozen or formalin fixed paraffin embedded tissue blocks. Quantification and quality assessment for RNA was performed with a Bioanalyzer (Agilent). Sequencing libraries were constructed from frozen tissue with a TruSeq mRNA library preparation kit using poly-A enriched RNA (Illumina). Capture enrichment was performed with the human kinome DNA capture baits (Agilent). Six libraries were pooled for each capture reaction, with 100 ng of each library, and custom blockers were added to prevent hybridization to adapter sequences.

Blocker B1:
5′-AGATCGGAAGAGCACACGTCTGAACTCCAGTCACNNNNNNATCTCGT
ATGCCGTCTTCTGCTTG/3′ddC
Blocker B2:
5′-CAAGCAGAAGACGGCATACGAGATNNNNNNGTGACTGGAGTTCAGAC
GTGTGCTCTTCCGATCT/3′ddC

Captured libraries were sequenced on an Illumina HiSeq2000 platform with a paired-end 51 base protocol. Sequences were aligned to the human genome

(Hg19) with TopHat (25). HTSeq was used to assess the number of uniquely assigned reads for each gene, expression values were then normalized to 107 total reads and log2 transformed. Sequence variants were detected with SAMtools and were annotated using the ENSEMBL variant effect predictor and the NHLBI GO Exome Variant Server.

Fusion Detection

Three platforms were used to identify and rank candidate fusion genes: TopHat-fusion (Kim and Salzberg, 2011. Genome Biol 12: R72 (2011); deFUSE (McPherson et al., 2011. PLoS Comput Biol 7, e1001138); and de novo transcript assembly with Trinity (Grabherr et al., 2011. Nature Biotech 29: 644. Detection parameters differed for each platform. For the de novo assembly approach, sequence reads where assembled using Trinity (version r2012-10-05) and the resulting transcripts were used to identify fusion candidates. The filtering pipeline consisted of a number of steps:

1. The de novo transcripts were aligned to the human genome (Hg19) RefSeq open reading frame sequences.

2. Transcripts were identified that had multiple RefSeq gene alignments.

3. Sequence reads were mapped back to the candidate fusion transcripts using bowtie2 (John Hopkins University; see bowtie-bio.sourceforge.net/bowtie2/index.shtml) to determine the number of spanning reads (reads aligning to the break-point with at least 15 bases on either size) and spanning pairs (pairs with a read on each side of the break-point).

4. Erroneous fusions were removed, for example, where the fusion partners shared sequence at the breakpoint, or no spanning read pairs were detected, or if the candidate fusion was identified in a normal sample.

Validation

The Maxima First Strand cDNA Synthesis Kit was used to produce input cDNA for RT-PCR (Thermo Scientific). PCR primers were designed to amplify across the fusion break points:

EML4-ALK
F: 5′-CACACCTGGGAAAGGACCTA
R: 5′-CACCTGGCCTTCATACACCT;
STRN-ALK
F: 5′-CACCTGGCCTTCATACACCT
R: 5′-AGAAAGGAAGGGCCAAGAAA;
FGFR3-TACC3
F: 5′-GACCGTGTCCTTACCGTGAC
R: 5′-CCTGTGTCGCCTTTACCACT;

wherein F denotes forward primer and R denotes Reversed primer.

Activating mutations in FGFR3 were verified by PCR amplification from cDNA using F:5′-CATTGGAGGCATAAGCTG and R:5′- AGCACGGTAACGTAGGGTGT) and capillary sequencing using the Big Dye Terminator V3.1 sequencing kit (Applied Biosystems).

Fluorescent In Situ Hhybridization (FISH)

ALK translocations were assessed using the Vysis ALKbreak-apart FISH probe kit (Abbott). Samples were processed according to the manufacturer's instructions (Vysis). In short, unstained FFPE sections (4 μm) were deparaffinized, treated with protease and washed in preparation for hybridization. FISH probes were hybridized for 14 to 24 hours at 37° C., after which the slides were washed thoroughly. Mounting medium with DAPI was added (Vector Laboratories) and coverslips were attached to facilitate imaging.

Immunohistochemistry

Immunohistochemistry was performed on the BenchMark Ultra automated staining instrument (Ventana Medical Systems). Paraffin sections (4 μm) were heated at 75° C. for 28 minutes and then deparaffinized in the instrument.

Sections were treated with CC1 buffer for 64 minutes before incubation with the primary antibody (Ventana Medical Systems).

For ALK staining, sections were incubated in a 1:50 dilution of the primary antibody (NCL-ALK, clone 5A4, Novocastra) for two hours at 37° C.

For FGFR3 staining, sections were incubated in a 1:50 dilution of the primary antibody (FGFR3, clone B-9, Santa Cruz) for 1 hour at room temperature followed by a Ventana amplification step (Ventana Medical Systems). Bound primary antibody was detected using the Universal DAB Detection Kit (Ventana Medical Systems) and slides were counterstained with Hematoxylin.

Results

Kinome-centred RNA sequencing identifies STRN as a novel ALK fusion partner A kinome-centred RNA sequencing method was developed in which biotinylated RNA probes are used to selectively capture kinase transcripts prior to sequencing. The capture increases the coverage of target transcripts and provides a more sensitive way to detect mutations. We began by looking for kinases that were involved in fusion genes in a panel of 95 NSCLCs, which included 36 adenocarcinomas, 48 squamous cell carcinomas (SCCs) and 11 others.

Hybridization to the probes targeting the human kinome resulted in an 18-fold enrichment in coverage for these transcripts. Three analysis platforms were employed to detect fusion transcripts, resulting in a list of 20 candidates (Table 2). Of these, 4 were also present in normal tissue and were not considered further.

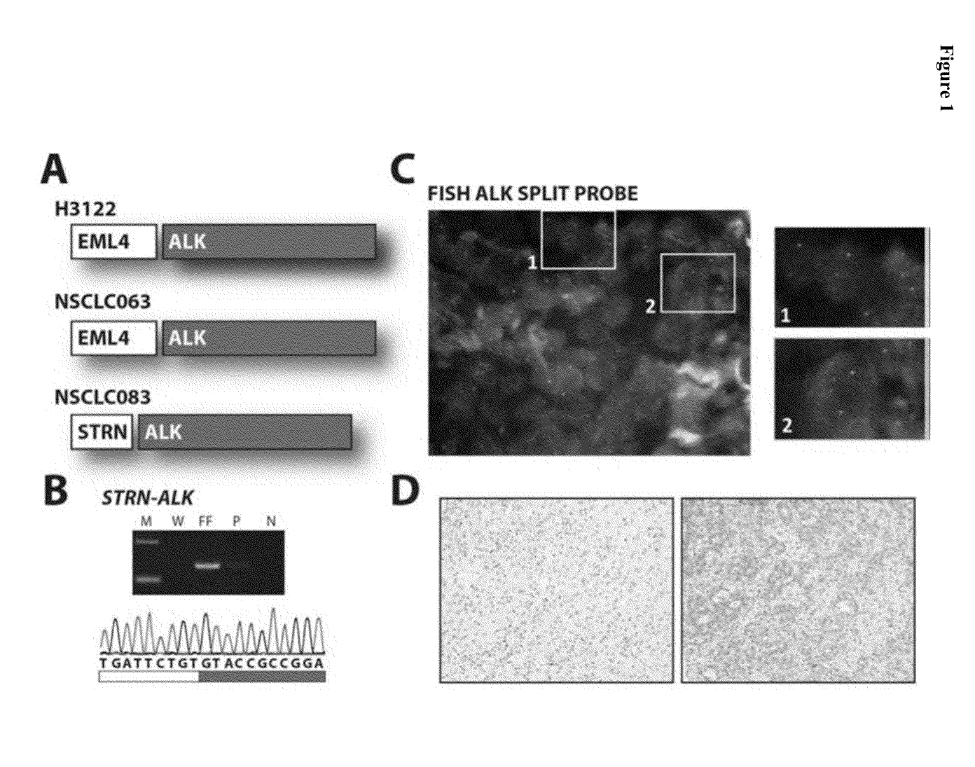

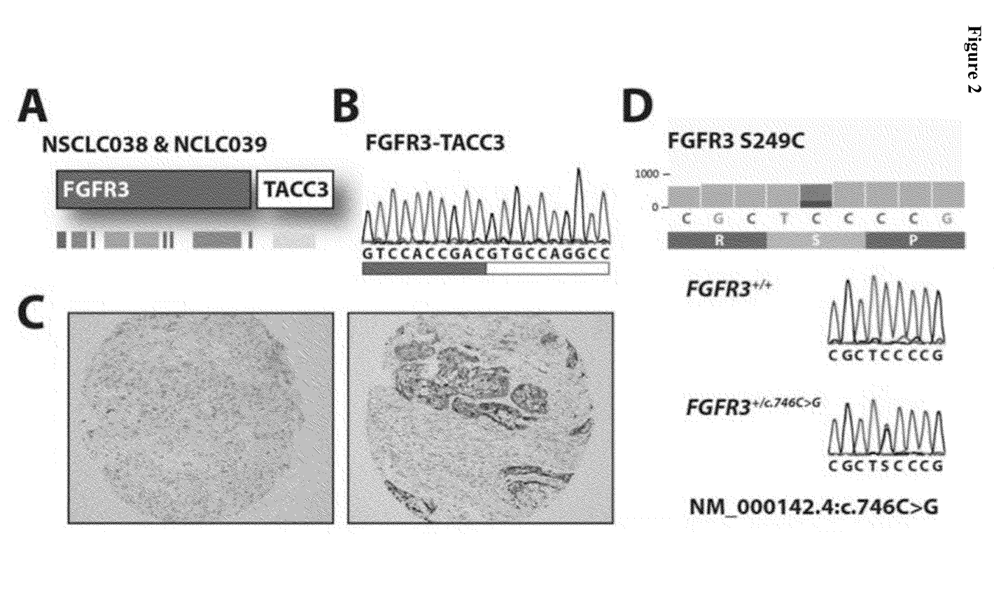

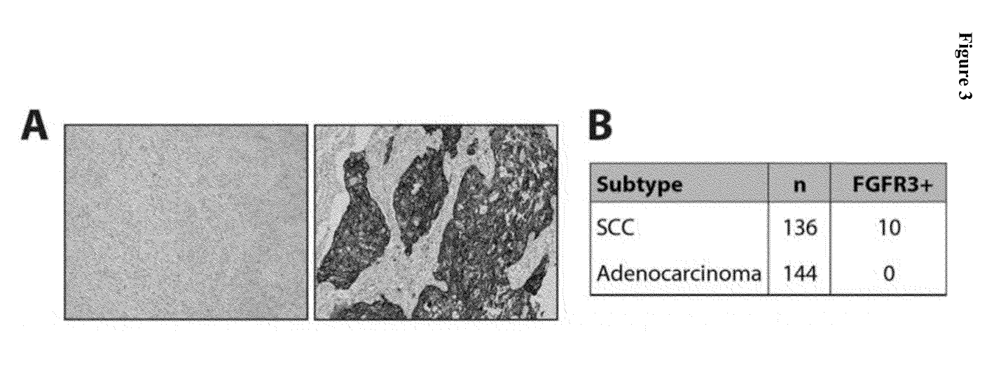

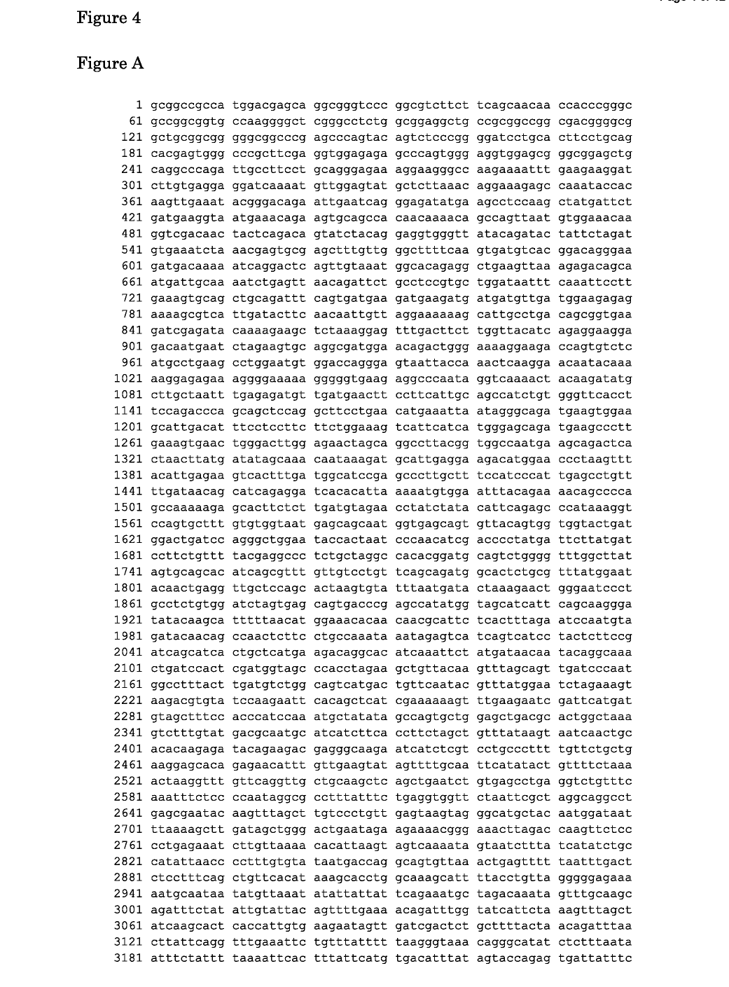

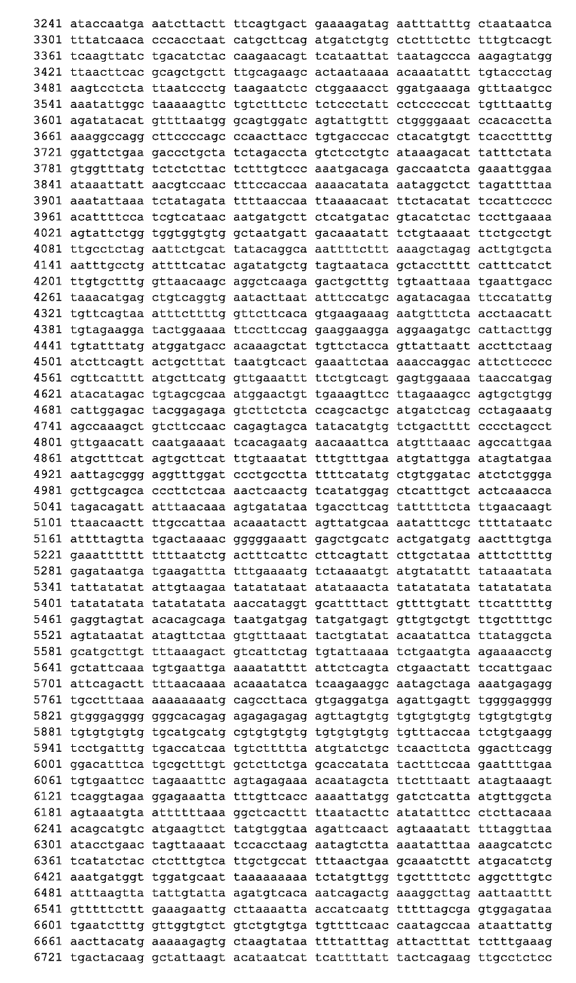

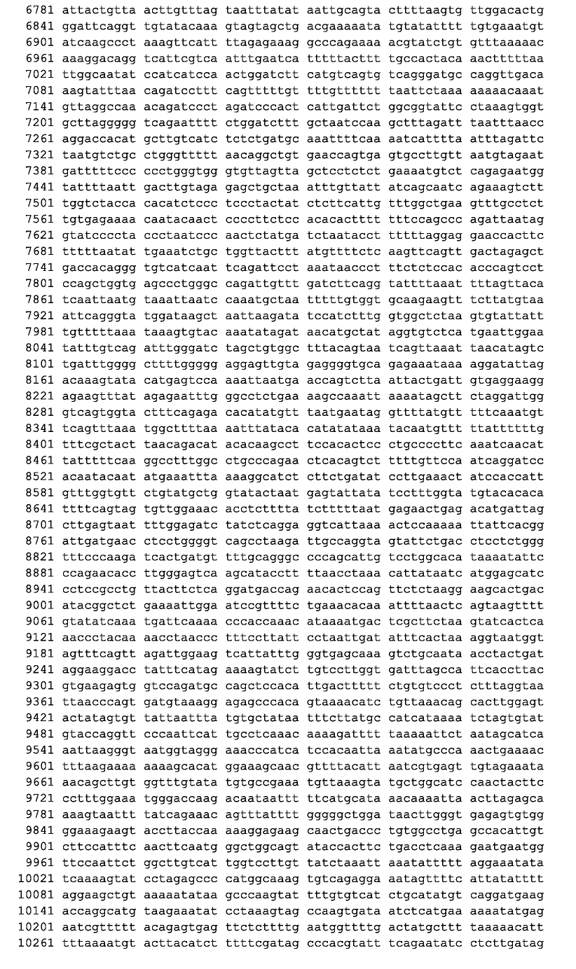

The EML4-ALK fusion was identified in one adenocarcinoma and in the H3122 cell line, which was included as a positive control. ALK was also found in another fusion, which joined exon 3 of striatin (STRN) to exon 20 of ALK. The STRN-ALK fusion produces an in-frame protein that contains the first 137 amino acids from STRN joined to the last 339 amino acids of ALK, a region that includes the kinase domain (FIG. 1). The EML4-ALK and STRN-ALK fusions were confirmed by RT-PCR with primers that spanned the breakpoint. STRN and ALK are both located on chromosome 2 but are separated by approximately 7 Mb; as the genes share the same transcriptional orientation it is most likely that the fusion results from a large intrachromosomal deletion. Rearrangement of the ALK locus was confirmed using FISH (FIG. 1C). The rearranged STRN-ALK gene produced two distinct signals in each nucleus, suggesting that the rearranged locus has also been amplified (FIG. 1C). Tumours that were positive for ALKfusions had the highest levels of ALK expression across the cohort (ranked 1 and 2 from a total of 95 samples). Staining with an antibody confirmed expression of ALK in the sample carrying the STRN-ALK fusion (FIG. 1D).

TABLE 2
Candidate fusions predicted by de novo assembly
High priority candidates were selected from the de novo assembly fusion detection method. Candidate fusions with more than 10 spanning pairs (read
pairs with one read on eitherside of the fusion boundary) are listed, unless the fusion was also identified in an unrelated normal sample. The distance
between the two genes is listed for intrachromosomal events (Gap, measured in kilo bases). Candidate fusions that were also detected with TopHat-
Fusion are marked (Y—yes). Each fusion transcript was assessed to determine if the transcript would produce an in-frame fusion (Y—yes, N—no).
PCR primers were designed to amplify candidate fusions from cDNA from the patients. Successful PCR of the fusion is noted in the table
(PCR, Y—yes, NT—not tested) and we confirmed the fusion event by capillary sequencing of the PCR products (Seq, Y—yes). Structural
variants involving RPS6KB1 have been described previously (Inaki et al., 2011. Genome Res 21: 676-87).

| | | | | | | Gap | Spanning | Spanning | Tophat | In | | |
|---|---|---|---|---|---|---|---|---|---|---|---|---|
| Sample | Fusion | Chr. | Base | Chr. | Base | (kb) | Pairs | Beads | Fusion | frame | PCR | Seq |
| NSCLC038 | FGFR3-TACC3 | 4 | 1739325 | 4 | 1808661 | 69 | 3375 | 348 | Y | Y | Y | Y |
| NSCLC033 | RPS6KB1-VMP1 | 17 | 57915556 | 17 | 57992064 | 76 | 274 | 90 | Y | N | Y | Y |
| H3122 | EML4-ALK | 2 | 29446394 | 2 | 42522656 | 13076 | 261 | 158 | Y | Y | Y | Y |
| NSCLC019 | IK2F3-NF1 | 17 | 29563039 | 17 | 37947669 | 8385 | 249 | 87 | | N | Y | |
| NSCLC063 | EML4-ALK | 2 | 29446394 | 2 | 42522655 | 13075 | 91 | 44 | Y | Y | Y | Y |
| NSCLC010 | PRCKZ-SK1 | 1 | 2106553 | 1 | 2161174 | 55 | 85 | 23 | Y | Y | | |
| NSCLC066 | FGGY TESK2 | 1 | 45887398 | 1 | 59922631 | 14035 | 84 | 47 | Y | Y | Y | Y |
| NSCLC039 | FGFR3-TACC3 | 4 | 1739325 | 4 | 1808661 | 69 | 36 | 5 | Y | Y | Y | Y |
| NSCLC055 | RPS5KB1-VMP1 | 17 | 57886157 | 17 | 58013902 | 128 | 32 | 40 | | Y | NT | |
| NSCLC010 | MCM5-TOM1 | 22 | 35741715 | 22 | 35819334 | 78 | 32 | 23 | Y | Y | Y | |
| NSCLC051 | MCM5-TRPM7 | 15 | 50940884 | 22 | 35817310 | | 30 | 32 | Y | N | NT | |
| NSCLC043 | LAMA3-RIOK3 | 18 | 21053393 | 18 | 21364121 | 311 | 30 | 16 | Y | N | Y | Y |
| NSCLC032 | AMPH-FDFT1 | 8 | 11696084 | 7 | 38574611 | | 30 | 11 | Y | Y | Y | Y |
| NSCLC024 | ATR-TOP3B | 3 | 142238511 | 22 | 22314108 | | 20 | 11 | Y | N | Y | Y |
| NSCLC012 | FTO-HERPUD1 | 16 | 54145674 | 16 | 56976149 | 2830 | 16 | 5 | Y | N | Y | Y |
| NSCLC010 | P2RX3-RTN4RL2 | 11 | 57135483 | 11 | 57235563 | 100 | 14 | 4 | | N | Y | |
| NSCLC035 | CHPT1-UTP20 | 12 | 101759237 | 12 | 102091922 | 333 | 13 | 12 | Y | Y | Y | Y |
| NSCLC028 | MSLN-WDR90 | 16 | 715679 | 16 | 814143 | 98 | 13 | 7 | | N | Y | |
| NSCLC083 | STRN-ALK | 2 | 29446394 | 2 | 37143221 | 7607 | 13 | 7 | | Y | Y | Y |

FGFR3 is recurrently mutated in squamous NSCLC

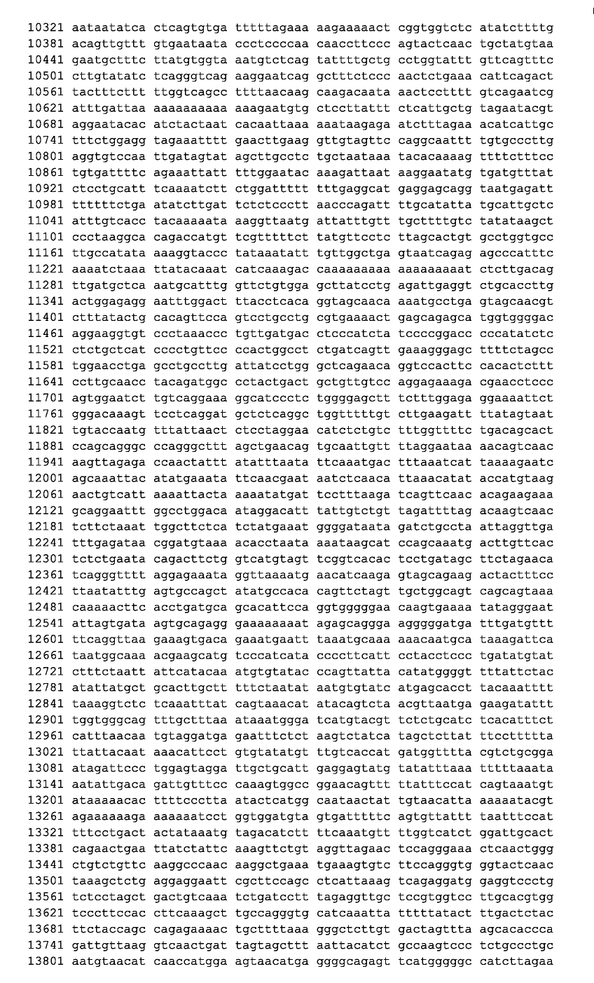

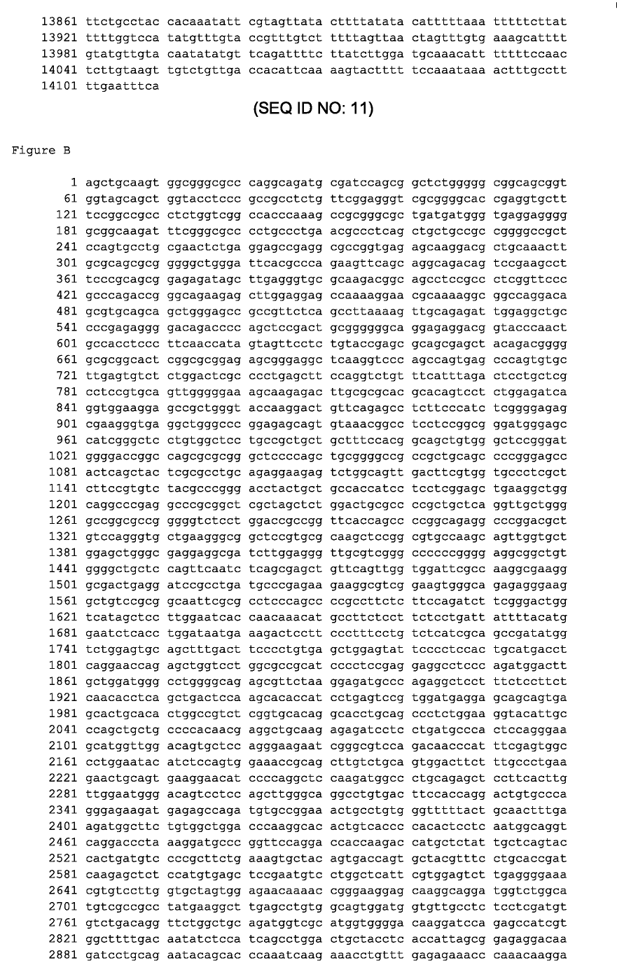

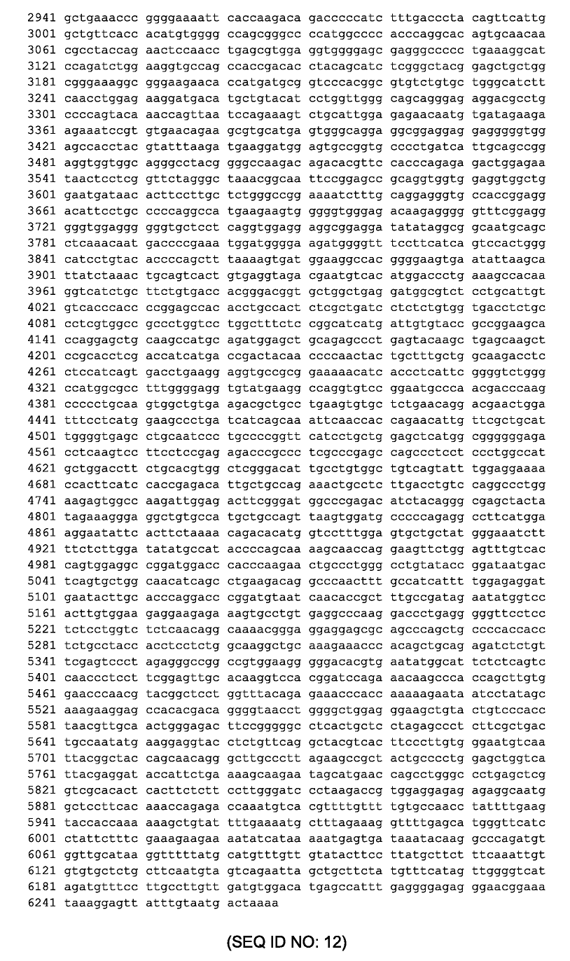

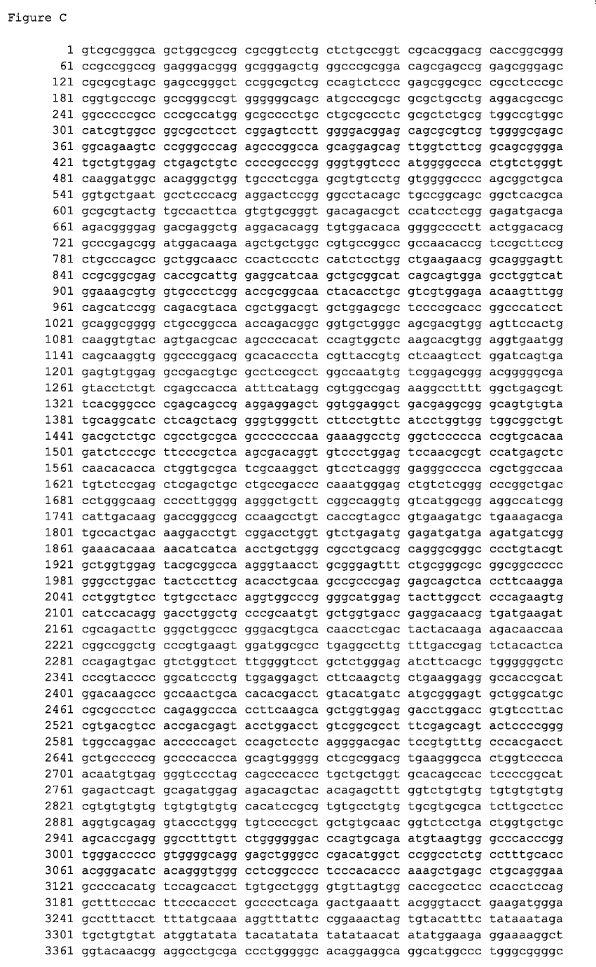

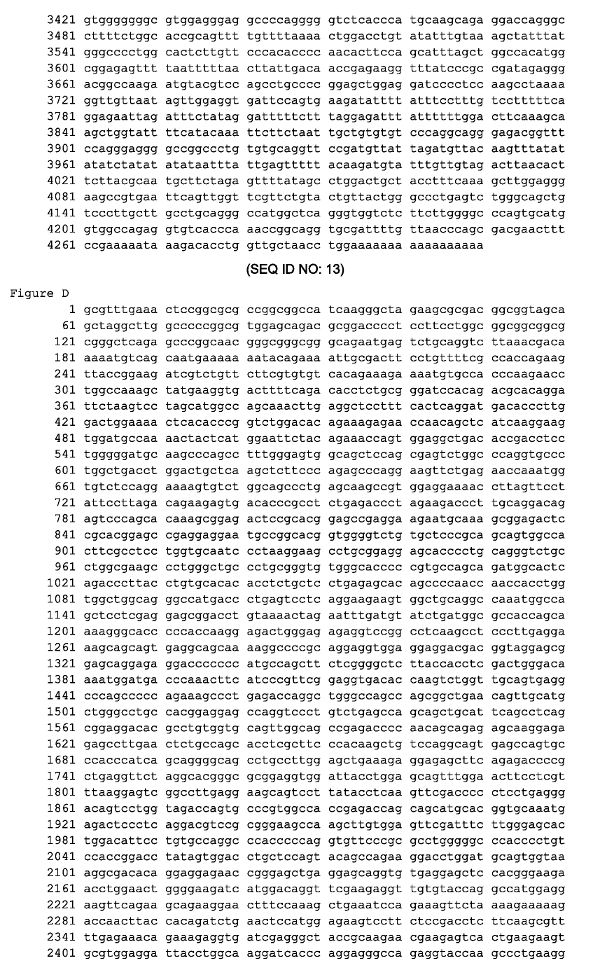

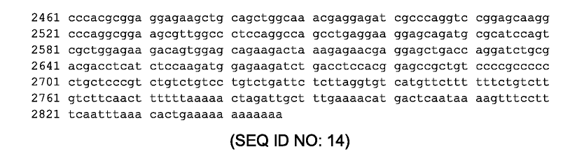

We also detected two SCC samples that carried a candidate fusion involving FGFR3 and transforming acidic coiled-coil containing protein 3 (TACC3). FGFR3-TACC3 fusions were recently identified in glioblastoma and bladder cancer (Parker et al., 2013. J Clin Invest 123: 855; Singh et al., 2012. Science 337: 1231); Williams et al., 2013. Hum Mol Genet 22: 795). The rearrangement places the first 18 exons of FGFR3, including almost the entire open reading frame, upstream of the last 7 exons of TACC3. The resulting fusion transcript is in frame, such that the last 226 amino acids of TACC3 are added directly to the truncated FGFR3 protein (amino acids 1-760). The C-terminus of the fusion protein includes a complete TACC domain (FIG. 2A). RT-PCR and capillary sequencing was used to confirm the fusion between exon 18 of FGFR3 and exon 10 of TACC3 (FIG. 2B). One patient had very low levels of the fusion transcript; to ensure that this was not due to contamination we confirmed the presence of the fusion using independent material derived from FFPE blocks. A diagnostic FISH assay used to detect FGFR3 translocations did not detect rearrangement at the locus, which reflects the fact that the two genes are separated by only 48 kilobases (data not shown).

Expression of the FGFR3 protein was markedly elevated in the samples that carried the FGFR3-TACC3 translocation (FIG. 2C). As well as detecting the gene fusion, we also identified two SCCs that carried activating mutations in FGFR3. The mutation causes a serine to cysteine substitution at position 249 and is the most common FGFR3 activating mutation identified in bladder cancer (FIG. 2D).

FGFR3 expression defines a subset of squamous NSCLC Expression of FGFR3 was assessed across a panel of 280 NSCLCs that included 136 squamous cell carcinomas and 144 adenocarcinomas. Strong FGFR3 expression was detected in 10 SCCs (7.4%), whereas the adenocarcinomas were uniformly negative (FIG. 3). Tumours that had high FGFR3 levels were screened by RT-PCR, which revealed two additional cases in which FGFR3 was fused to TACC3.

1. A method for determining the presence or absence of striatin-anaplastic lymphoma kinase (STRN-ALK) gene fusion and/or a Fibroblast Growth Factor Receptor 3—transforming acidic coiled-coil containing protein 3 (FGFR3-TACC3) gene fusion in an individual, said method comprising:
a) evaluating a relevant nucleic acid sample of said individual to determine whether a portion of STRN nucleic acid is adjacent to a portion of ALK nucleic acid on a single polynucleotide and/or whether a portion of FGFR3 nucleic acid is adjacent to a portion of TACC3 nucleic acid on a single polynucleotide; and
b) identifying said individual as having a STRN-ALK gene fusion when a portion of the STRN nucleic acid is adjacent to a portion of the ALK nucleic acid on a single polynucleotide and/or as having a FGFR3-TACC3 gene fusion when a portion of the FGFR3 nucleic acid is adjacent to a portion of the TACC3 nucleic acid on a single polynucleotide.

2. The method according to claim 1, wherein said portion of the STRN gene comprises a caveolin binding domain-encoding region and a coiled coil encoding region and/or said portion of the FGFR3 gene comprises a kinase encoding domain.

3. The method according to claim 1, wherein said portion of ALK nucleic acid comprises a kinase-encoding region and/or wherein said portion of TACC3 nucleic acid comprises a coiled-coil encoding region.

4. The method according to claim 1, wherein the nucleic acid sample that is evaluated from said individual comprises genomic DNA or mRNA.

5. The method according to claim 1, wherein said method comprises amplification of at least part of the nucleic acid.

6. The method according to claim 5, wherein said method comprises the use of a primer pair comprising the nucleotide sequence of SEQ ID NO: 1 and of SEQ ID NO: 2.

7. The method according to claim 5, wherein said method comprises the use of a primer pair comprising the nucleotide sequence of SEQ ID NO: 3 and of SEQ ID NO: 4.

8. The method according to claim 5, wherein said amplification is by PCR.

9. The method according to claim 1, wherein said method comprises detecting said gene fusion by hybridizing a probe encompassing a first portion that is specific for STRN nucleic acid and a second portion that is specific for ALK nucleic acid and/or a probe encompassing a first portion that is specific for FGFR3 nucleic acid and a second portion that is specific for TACC3 nucleic acid.

10. The method according to claim 9, wherein said probe comprises the nucleotide sequence of SEQ ID NO: 5 and/or of SEQ ID NO: 6.

11. The method according to claim 5, further comprising determining the presence or absence of said gene fusion by determining the nucleotide sequence of the amplified nucleic acid.

12. The method according to claim 5, wherein said method comprises determining the presence or absence of said gene fusion by determining the size of the amplified nucleic acid.

13. A method for diagnosing an individual as having adenocarcinoma, said method comprising determining the presence or absence of STRN-ALK gene fusion and/or of FGFR3-TACC3 gene fusion according to the method of claim 1; and:
diagnosing said individual as having adenocarcinoma when said STRN-ALK gene fusion is present and/or diagnosing said individual as having squamous cell carcinoma (SCC) when said FGFR3-TACC3 gene fusion is present.

14. A method for treating an individual suffering from adenocarcinoma, said method comprising
diagnosing an individual as having adenocarcinoma according to the method of claim 13; and
treating said individual with an ALK inhibitor.

15. A method for treating an individual suffering from SCC, said method comprising
diagnosing an individual as having SCC according to the method of claim 13; and
treating said individual with an inhibitor of FGFR3.
